(19) United States
(12) Patent Application Publication
(10) Pub. No.: US 2025/0059466 A1
(43) Pub. Date: Feb. 20, 2025

(54) PROCESS FOR MAKING GRANULES AND POWDERS

(71) Applicant: BASE SE, Ludwigshafen Am Rhein (DE)

(72) Inventors: Bernd HINRICHSEN, Ludwigshafen (DE); Martin VIERTELHAUS, Ludwigshafen (DE); Michael Klemens MUELLER, Ludwigshafen (DE); Matthias VOGES, Muenster (DE); Astrid SCHMIDT, Ludwigshafen (DE); Matthias ARNDT, Ludwigshafen (DE)

(21) Appl. No.: 18/721,104

(22) PCT Filed: Dec. 8, 2022
(86) PCT No.: PCT/EP2022/084937

§ 371 Date: Jun. 17, 2024

(30) Foreign Application Priority Data
Dec. 17, 2021 (EP) 21215601.2

(51) Int. Cl. C11D 3/33; C07C 227/16; C11D 11/00; C11D 17/06

(52) U.S. Cl. CPC C11D 3/33; C07C 227/16; C11D 11/0082; C11D 17/06; C11D 2111/14

(57) ABSTRACT

Process for making a modified granule or powder comprising an alkali metal salt of an aminocarboxylate complexing agent (A), said process comprising the steps of

(a) providing a granule or powder comprising an alkali metal salt of an aminocarboxylate complexing agent (A),
(b) treating said granule or powder with the respective aminocarboxylate complexing agent (A) in which at least one amino group or carboxyl group per molecule is present in the acid form, in the presence or absence of water,
(c) performing a heat treatment at a temperature in the range of from 80 to 150° C.

The present invention is directed to a process for making a modified granule or powder comprising an alkali metal salt of an aminocarboxylate complexing agent (A), said process comprising the steps of
(a) providing a granule or powder comprising an alkali metal salt of an aminocarboxylate complexing agent (A),
(b) treating said granule or powder with the respective aminocarboxylate complexing agent (A) in which at least one amino group or carboxyl group per molecule is present in the acid form in the presence or absence of water,
(c) performing a heat treatment at a temperature in the range of from 80 to 150° C.

Chelating agents of the aminocarboxylate type such as methyl glycine diacetic acid (MGDA) and glutamic acid diacetic acid (GLDA) and their respective alkali metal salts are useful sequestrants for alkaline earth metal ions such as Ca²⁺ and Mg²⁺. A lot of aminocarboxylates show good biodegradability and are thus environmentally friendly. For that reason, they are recommended and used for various purposes such as laundry detergents and for automatic dishwashing (ADW) formulations, in particular for so-called phosphate-free laundry detergents and phosphate-free ADW formulations.

Depending on the type of product—liquid home care and fabric care products versus solid home care and fabric care products—and the manufacturing process of solid home care and fabric care products care product manufacturers may either prefer to handle solutions of aminocarboxylates or solid aminocarboxylates, for example joint spray drying or solid mixing. Powders and granules of aminocarboxylates may be shipped economically due to their high active ingredient content that goes along with low water content. Therefore, convenient processes for providing granules are still of great commercial interest.

In WO 2009/103822, a process is disclosed in which slurries are granulated that have a certain solids content, with a gas inlet temperature of 120° C. or less. In WO 2012/168739, a process is disclosed wherein slurries of complexing agents are spray-dried under non-agglomerating conditions.

Both processes have their shortcomings. A low gas inlet temperature requires highly concentrated slurries or a huge amount of gas per unit of granule. A process using non-agglomerating conditions provides for powders only.

An ongoing challenge in the context with aminocarboxylate chelating agents is yellowing and stability in the presence of peroxides of powders and of granular materials. An additional problem may be water uptake upon storage. Although various suggestions have been made, e.g., WO 2015/121170, there is still ongoing research to further improve the yellowing.

It was therefore an objective of the present invention to provide a process that allows to produce granules and powders of aminocarboxylate complexing agents at an acceptable water uptake and with a reduced tendency to yellowing and an improved storage stability in the presence of peroxides.

Accordingly, the process as defined at the outset has been defined, hereinafter also referred to as inventive process or as process according to the present invention. The inventive process comprises three mandatory steps, step (a), step (b), and step (c). They may in brief also be referred to as (a), (b), or (c), respectively. Steps (b) and (c) may be performed simultaneously, or steps (a), (b), and (c) are performed subsequently. Steps (a) to (c) are described in more detail below.

In step (a), a granule or powder comprising an alkali metal salt of an aminocarboxylate complexing agent (A) is provided, said hereinafter also referred to as “granule (A)” or “powder (A)”, respectively. In this context, alkali metal salts are selected from lithium salts, sodium salts, potassium salts, rubidium salts, and cesium salts and combinations of at least two of the foregoing, with potassium salts being preferred and sodium salts being more preferred.

Examples of aminocarboxylate complexing agents (A) are iminodisuccinates, and diacetates of amino acids, especially alanine, glutamic acid, and aspartic acid.

Preferably, complexing agent (A) is selected from methylglycine diacetic acid (MGDA) and glutamic acid diacetic acid (GLDA). Even more preferably, complexing agent (A) is selected from MGDA.

Complexing agent (A) may refer to fully neutralized aminocarboxylate complexing agents (A) and to partially neutralized aminocarboxylate complexing agents (A).

In one embodiment of the present invention, complexing agent (A) is selected from compounds according to general formula (I)

[CH₃—CH(COO)—N(CH₂—COO)₂]M₃₋ₓHₓ (I)

wherein

M is selected from alkali metal cations, same or different, preferably K or Na or combinations thereof, and even more preferably Na, and

x is in the range of from zero to 1.0, preferably zero to 0.3.

In any way, complexing agent (A) may bear a cation other than alkali metal. It is thus possible that minor amounts, such as 0.01 to 5 mol-% of total MGDA, respectively, bear alkali earth metal cations such as Mg²⁺ or Ca²⁺, or an Fe²⁺ or Fe³⁺ cation.

Complexing agents (A) such as MGDA and GLDA are selected from the racemic mixtures, the D-isomers and the L-isomers, and from mixtures of the D- and L-isomers other than the racemic mixtures. Preferably, complexing agent (A) is MGDA-Na₃ selected from the racemic mixture and from mixtures containing in the range of from 51 to 95 mole-% of the L-isomer, the balance being D-isomer. Particularly preferred are complexing agents (A) selected from the racemic mixture and mixtures of the enantiomers of MGDA-Na₃ with predominantly the L-enantiomer with an ee value in the range of from 0.1% or from 0.5% to 30%. Other particularly preferred embodiments are racemic mixtures.

In one embodiment of the present invention, granules (A) have an average diameter (D50) in the range of from 150 μm to 1.5 mm, preferably from 500 μm to 1.25 mm. The average diameter of granules may be determined, e.g., by optical or preferably by sieving methods, and refers to the volume average. Sieves employed may have a mesh in the range of from 60 to 1,250 μm.

In one embodiment of the present invention, the average particle diameter (D50) increases by 20 to 100 μm in the course of step (b), preferably 40 to 80 μm.

In one embodiment of the present invention, powders (A) have an average particle diameter (D50) in the range of from 1 to 120 μm, preferably 10 to 100 μm. The average particle diameter of powders can be determined, e.g., by LASER diffraction methods, for example with Malvern apparatus, and refers to the volume average.

Said granule (A) or powder (A) may comprise one or more ingredients other than complexing agent (A), for example a (co)polymer of (meth)acrylic acid. Examples of such granules and powders are disclosed, e.g., in WO 2015/121170.

Said granule may essentially contain only salt (A) and moisture. In other embodiments, said granule may contain one or more additives, for example up to 20% by weight, preferably 7 to 14% by weight, referring to salt (A). Examples of additives are silicates, especially sodium silicate, citrates, especially sodium citrate, polyvinylalcohol, and (co)polymers other than polyvinylalcohol, hereinafter also referred to as (co)polymers (C).

(Co)polymer (C) is selected from polymers (C) of (meth)acrylic acid and of copolymers (C) of (meth)acrylic acid, preferably of acrylic acid, partially or fully neutralized with alkali. In the context of the present invention, copolymers (C) are those in which at least 50 mol-% of the comonomers are (meth)acrylic acid, preferably at least 75 mol-%, even more preferably 80 to 99 mol-%.

Suitable comonomers for copolymers (C) are ethylenically unsaturated compounds, such as styrene, isobutene, ethylene, α-olefins such as propylene, 1-butylene, 1-hexene, and ethylenically unsaturated dicarboxylic acids and their alkali metal salty and anhydrides such as but not limited to maleic acid, fumaric acid, itaconic acid disodium maleate, disodium fumarate, itaconic anhydride, and especially maleic anhydride. Further examples of suitable comonomers are C₁-C₄-alkyl esters of (meth)acrylic acid, for example methyl acrylate, methyl methacrylate, ethyl acrylate, ethyl methacrylate, n-butyl acrylate.

In one embodiment of the present invention, (co)polymer (C) is selected from copolymers of (meth)acrylic acid and a comonomer bearing at least one sulfonic acid group per molecule. Comonomers bearing at least one sulfonic acid group per molecule may be incorporated into copolymer (C) as free acid or least partially neutralized with alkali. Particularly preferred sulfonic-acid-group-containing comonomers are 1-acrylamido-1-propanesulfonic acid, 2-acrylamido-2-propanesulfonic acid, 2-acrylamido-2-methylpropanesulfonic acid (AMPS), 2-methacrylamido-2-methylpropanesulfonic acid, 3-methacrylamido-2-hydroxypropanesulfonic acid, allylsulfonic acid, methallylsulfonic acid, allyloxybenzenesulfonic acid, methallyloxyben-zenesulfonic acid, 2-hydroxy-3-(2-propenyloxy)propanesulfonic acid, 2-methyl-2-propene-1-sulfonic acid, styrenesulfonic acid, vinylsulfonic acid, 3-sulfopropyl acrylate, 2-sulfoethyl methacrylate, 3-sulfopropyl methacrylate, sulfomethacrylamide, sulfomethylmethacrylamide, and salts of said acids, such as the sodium salts, potassium salts or ammonium salts thereof.

Copolymers (C) may be selected from random copolymers, alternating copolymers, block copolymers and graft copolymers, alternating copolymers and especially random copolymers being preferred.

Useful copolymers (C) are, for example, random copolymers of acrylic acid and methacrylic acid, random copolymers of acrylic acid and maleic anhydride, ternary random copolymers of acrylic acid, methacrylic acid and maleic anhydride, random or block copolymers of acrylic acid and styrene, random copolymers of acrylic acid and methyl acrylate. More preferred are homopolymers of methacrylic acid. Even more preferred are homopolymers of acrylic acid.

(Co)polymer (C) may constitute straight-chain or branched molecules. Branching in this context will be when at least one repeating unit of such polymer (C) is not part of the main chain but forms a branch or part of a branch. Preferably, polymer (C) is not cross-linked.

In one embodiment of the present invention, (co)polymer (C) has an average molecular weight M_{w} in the range of from 1,200 to 30,000 g/mol, preferably from 2,500 to 15,000 g/mol and even more preferably from 3,000 to 10,000 g/mol, determined by gel permeation chromatography (GPC) and referring to the respective free acid.

(Co)polymer (C) is partially neutralized with alkali, for example with lithium or potassium or sodium or combinations of at least two of the forgoing, especially with sodium. For example, in the range of from 10 to 95 mol-% of the carboxyl groups of (co)polymer (C) may be neutralized with alkali, especially with sodium, preferably in the range from 20 to 70 mole-% of the carboxyl groups, even more preferred in the range of from 25 to 60 mole-%.

In one embodiment of the present invention, copolymer (C) is selected from a combination of at least one polyacrylic acid and at least one copolymer of (meth)acrylic acid and a comonomer bearing at least one sulfonic acid group per molecule, both polymers being partially neutralized with alkali.

In one embodiment of the present invention, (co)polymer (C) is selected from sodium salts of polyacrylic acid with an average molecular weight M_{w} in the range of from 1,200 to 30,000 g/mol, preferably from 2,500 to 15,000 g/mol and even more preferably from 3,000 to 10,000 g/mol, determined by gel permeation chromatography (GPC) and referring to the respective free acid.

If addition of a (co)polymer (C) or any of the aforementioned additives is desired, said (co)polymer (C) may be added when manufacturing said granule (A) or powder (A). In one embodiment, granule (A) or powder (A), respectively, do not contain any (co)polymer of (meth)acrylic acid.

Preferably, particles of said granule (A) or powder (A) have a spherical form.

Processes for manufacture of granules (A) and powders (A) are known per se.

In step (b), said granule (A) or powder (A) is treated with the respective aminocarboxylate complexing agent (A) in which at least one amino group or at least one carboxyl group per molecule is present in the acid form—hereinafter also referred to as complexing agent (B), in the absence or—preferably—in the presence of water. In embodiments where the so-called free acid is used, two or three carboxyl groups and the amino group are protonated and thus in the acid form. In embodiments wherein granule (A) or powder (A) contains a partially acidified (neutralized) salt of aminocarboxylate complexing agent (A), the degree of acidity of aminocarboxylate complexing agent (B) applied in step (b) is higher in starting material (A).

Such complexing agent (B) may be obtained by acidifying the respective alkali metal salts of aminocarboxylate complexing agents (A) with an inorganic acid, for example sulfuric acid or hydrochloric acid, or by ion exchange. The free acid of, e.g., MGDA may be obtained by neutralization of MGDA-Na₃ with gaseous HCl or with H₂SO₄.

Inorganic acids are selected from inorganic compounds with at least one acidic proton, for example with a pKₐ value of 12 or less, preferably of 5 or less. Examples are Brönsted acids with one, two or three acidic protons, and mono-alkali metal salts of di-or tribasic inorganic acids. Specific examples of inorganic acids are HNO₃, HCl, H₂SO₄, NaHSO₄, KHSO₄, preferred are H₂SO₄, NaHSO₄, and more preferred is H₂SO₄. For making the free acid of aminocarboxylate complexing agent (A) and especially of MGDA, HCl and sulfuric acid are preferred.

Suitable organic acids bear two or three carboxylic acid groups. Examples are citric acid, tartaric acid, adipic acid, glutamic acid, succinic acid, malic acid, and ascorbic acid, with adipic acid and citric acid being preferred and citric acid even being more preferred. Monocarboxylic acids may have a strong smell when in the form of their free acid.

Neutralization reactions of aminocarboxylate complexing agents (A) with inorganic acids are known per se. They may be performed at a temperature in the range of from 5 to 50° C. or even higher, preferred is ambient temperature.

Step (b) may be performed in the absence or—preferably—in the presence of water. In this context, the absence of water means that aminocarboxylate complexing agent (A) in which at least one carboxyl group is present in the acid form is applied as a solid, and water of crystallization is neglected as well as any moisture content of the respective granule or powder. In such embodiments, step (b) may be carried out in a mixer.

However, it is preferred to perform step (b) in the presence of water, for example, step (b) is in performed in a fluidized bed or in an essentially horizontal cylindrical drying apparatus containing a stirring element that rotates around an essentially horizontal axis.

In one embodiment of the present invention, a spray granulator is charged with a granule of aminocarboxylate complexing agents (A) which is then fluidized, thereby obtaining a fluidized bed. Then, an aqueous solution or slurry of complexing agent (B) is sprayed into the fluidized bed, and the water is removed by evaporation at least partially, for example 80 to 99% of the water of said solution or slurry.

In one embodiment of the present invention, the bed temperature is maintained in the range of from 80 to 125° C.

In one embodiment of step (b), step (b) is performed in an essentially horizontal cylindrical drying apparatus containing a stirring element that rotates around an essentially horizontal axis, for example in a continuous fluidization technology dryer (“CFT”). Such dryers are known and commercially available from Buss SMS Canzler GmbH.

In one embodiment of the present invention, said aqueous solution or slurry of complexing agent (B) is introduced into a cylindrical drying apparatus that is charged with granule or powder (A), for example to a level of filling in the range of from 50 to 90%, preferably 60 to 80%. Such granule or has an average diameter similar to the desired diameter of the granule to be manufactured, for example 75 to 95% of the specified diameter.

Said introducing may be performed through one or more dosing lances or nozzles, for example single-fluid nozzles and two-fluid nozzles, two-fluid nozzles being preferred. In such embodiments, the first fluid is the aqueous solution or slurry of complexing agent (B), and the second fluid is compressed gas, for example with a pressure of 1.1 to 7 bar. Said compressed gas may have a temperature in the range of from 100 to 250° C., preferably 125 to 220° C. The introduction of solution preferably occurs from the top of the cylindrical drying apparatus.

In one embodiment, the feed of aqueous solution or slurry of complexing agent (B) is introduced using one or more extruders. If one or more extruders are applied, fines from an optional milling and sieving step can be introduced trough this extruder as well.

In one embodiment of the present invention, the solution or slurry of complexing agent (B) is heated to 115 to 150° C. and under elevated pressure and then introduced into said cylindrical drying apparatus in accordance to step (b), preferably with a one-component nozzle. Preferably, the respective solution or slurry is heated to 70 to 107° C. under ambient pressure and then introduced into said cylindrical drying apparatus in accordance to step (b), preferably with a one-component nozzle.

In step (c), most of the water is removed by evaporation. Granule or powder made by the inventive process may, when removed, see step (c), still contain residual moisture, for example 1 to 20% by weight, preferably 7 to 14% by weight, referring to the solids content, determined by drying at 200° C. for 1 hour at a Thermo balance, for example the METTLER-TOLEDO HX204.

In embodiments where steps (b) and (c) are performed simultaneously, the below applies to step (c) as well.

The pressure during step (b) may be normal pressure or lower. In one embodiment of the present invention, the pressure during step (b) is in the range of from 100 to 600 mbar abs in the cylindrical drying apparatus. For technical reasons, 100 to 200 mbar abs are preferred.

In one embodiment of the present invention, the temperature of the gas inside the cylindrical vessel is in the range of from 70 to 150° C., preferably 80 to 120° C. and even more preferably 100 to 115° C.

In one embodiment of the present invention, during step (b) the rotation speed of the stirring element is in the range of from 10 to 300 revolutions per minute (rpm), preferred in production scale 30 to 180, even more preferred 30 to 80. Depending on the diameter of the cylindrical vessel and of the stirring element, e.g., the stirring paddle or stirring blade, that results in a tip velocity of0.8 to 20 m/s.

In another embodiment of the present invention, step (b) is performed in a spouted bed.

In one embodiment of the present invention, aminocarboxylate complexing agents (A) and complexing agent (B) are applied in a molar ratio in the range of from 100:1 to 1:1, preferably from 50:1 to 5:1 and more preferably from 20:1 to 10:1.

In one embodiment of the present invention, the residence time in step (b) is in the range of from 10 minutes to 12 hours.

In step (c), a heat treatment at a temperature in the range of from 80 to 150° C. is performed. Said heat treatment may be performed simultaneously or subsequently to step (b). The temperature refers to the temperature of the solid, for example in a bed of granules.

In one embodiment of the present invention, a sieving step is performed subsequently to step (c). Said—optional—sieving step may be used for removing fines and lumps from the granule. Said lumps to be separated off are particles that may have a minimum particle diameter of 150% of the desired diameter, for example, 1,500 μm to 5 mm or even more depending on the desired particle diameter. Fines may have an average diameter in the range from particles diameter in the range of from 1 to 150 μm. The desired diameter corresponds to the respective specified diameter of potential customers.

Further—optional—steps are, e.g., rounding steps and cooling steps. In one embodiment, the resulting granules are rounded to increase bulk density. In one embodiment, an additional downstream drying step (post drying) is applied. In one embodiment, the granules are cooled down to improve storage behavior.

By the inventive process, a granule or powder with excellent properties may be obtained. The term “granule” in the context of the present invention refers to particulate materials that are solids at ambient temperature and that preferably may have an average particle diameter (D50) in the range of from 150 μm to 2 mm, preferably 0.4 mm to 1.25 mm, even more preferably 400 μm to 1 mm. The average particle diameter of inventive granules can be determined, e.g., by optical or preferably by sieving methods. Sieves employed may have a mesh in the range of from 60 to 3,000 μm.

Especially, granules obtained according the inventive process are less hygroscopic and give no or little raise to yellowing upon contact with percarbonate. They can therefore be used favourably in cleaners such as laundry detergents and automatic dishwashing detergents, for example in the form of powders or tabs, in combination with bleaching agents such as peroxides and percarbonates.

A further aspect of the present invention is related to granules or powders, hereinafter also referred to as inventive granules or inventive powders, respectively. Inventive granules or inventive powders comprise aminocarboxylate complexing agent and they have a core and a surface wherein said granule or powder has a pH value gradient, the pH value being higher in the core than at the surface, for example by 2 to 8 units.

In one embodiment of the present invention, inventive granules and inventive powders are solid alkali metal salts (A) of an aminocarboxylate complexing agent wherein (A) in the core is selected from compounds according to general formula (I)

[CH₃—CH(COO)—N(CH₂—COO)₂]M₃₋ₓHₓ (I)

wherein

M is selected from alkali metal cations, same or different, and

x is in the range of from zero to 0.3

and complexing agent (B) at the surface is selected from the same compound as aminocarboxylic acid (A) but with x being in the range of from 1.0 to 3.0.

In one embodiment of the present invention, inventive granule has an average particle diameter (d50) in the range of from 150 μm to 1.5 mm.

In one embodiment of the present invention, inventive powder has an average particle diameter (d50) in the range of from 50 μm to 100 μm.

The average particle diameter of inventive granules can be determined, e.g., by optical or preferably by sieving methods.

In one embodiment of the resent invention, the XRD pattern of said powder or granule exhibits a peak at 2Θ=9.1 and a duplet peak at 2Θ 14.23 and 14.95, or peaks at 2Θ=6.98, 13.99 and 17.77, or peaks at 2Θ=14.06 and 18.41 and 26.82, determined with Cu—Kα radiation (1.54 Å).

In one embodiment of the present invention, the XRD pattern of said powder or granule exhibits a peak at 2Θ=9.1 and a duplet peak at 2Θ 14.23 and 14.95, and peaks at 2Θ=6.98, 13.99 and 17.77, determined with Cu—Kα radiation (1.54 Å).

n one embodiment of the present invention, the XRD pattern of said powder or granule exhibits peaks at 2Θ=6.98, 13.99 and 17.77 and peaks at 2Θ=14.06 and 18.41 and 26.82, determined with Cu—Kα radiation (1.54 Å).

Inventive powders and especially inventive granules show excellent properties especially with respect to yellowing and hygroscopicity

Another aspect of the present invention relates to the use of inventive granules and inventive powders, and another aspect of the present invention relates to methods of use inventive granules and inventive powders. The preferred use of inventive granules and inventive powders is for the manufacture of solid laundry detergent compositions and of solid detergent compositions for hard surface cleaning, especially of solid automatic dishwashing detergents. Solid laundry detergent compositions and solid detergent compositions for hard surface cleaning may contain some residual moisture, for example 0.1 to 10% by weight, but are otherwise solid mixtures in the form of, e.g., powders, granules or tablets. The residual moisture content may be determined, e.g., by drying under vacuum at 80° C. Another aspect of the present invention relates to solid laundry detergent compositions and to solid detergent compositions for hard surface cleaning.

In the context of the present invention, the term “detergent composition for cleaners” includes cleaners for home care and for industrial or institutional applications. The term “detergent composition for hard surface cleaners” includes compositions for dishwashing, especially hand dishwash and automatic dishwashing and ware-washing, and compositions for other hard surface cleaning such as, but not limited to compositions for bathroom cleaning, kitchen cleaning, floor cleaning, descaling of pipes, window cleaning, car cleaning including truck cleaning, furthermore, open plant cleaning, cleaning-in-place, metal cleaning, disinfectant cleaning, farm cleaning, high pressure cleaning, but not laundry detergent compositions.

In the context of the present invention and unless expressly stated otherwise, percentages in the context of ingredients of laundry detergent compositions are percentages by weight and refer to the total solids content of the respective laundry detergent composition. In the context of the present invention and unless expressly stated otherwise, percentages in the context of ingredients of detergent composition for hard surface cleaning are percentages by weight and refer to the total solids content of the detergent composition for hard surface cleaner.

In one embodiment of the present invention, solid laundry detergent compositions according to the present invention may contain in the range of from 1 to 30% by weight of inventive granule or inventive powder. Percentages refer to the total solids content of the respective laundry detergent composition.

In one embodiment of the present invention, inventive solid detergent compositions for hard surface cleaning may contain in the range of from 1 to 50% by weight of inventive granule or inventive powder, preferably 5 to 40% by weight and even more preferably 10 to 25% by weight. Percentages refer to the total solids content of the respective detergent composition for hard surface cleaning.

Particularly advantageous inventive solid detergent compositions for hard surface cleaning and inventive solid laundry detergent compositions, especially for home care, contain one or more complexing agent other than inventive granule or inventive powder, respectively. Inventive solid detergent compositions for hard surface cleaning and inventive solid laundry detergent compositions may contain one or more complexing agent (in the context of the present invention also referred to as sequestrant) other than from inventive granule or powder. Examples are citrate, phosphonic acid derivatives, for example the disodium salt of hydroxyethane-1,1-diphosphonic acid (“HEDP”), and polymers with complexing groups like, for example, polyethylenimine in which 20 to 90 mole-% of the N-atoms bear at least one CH₂COO⁻ group, and their respective alkali metal salts, especially their sodium salts, for IDS-Na₄, and trisodium citrate, and phosphates such as STPP (sodium tripolyphosphate). Due to the fact that phosphates raise environmental concerns, it is preferred that advantageous detergent compositions for cleaners and advantageous laundry detergent compositions are free from phosphate. “Free from phosphate” should be understood in the context of the present invention, as meaning that the content of phosphate and polyphosphate is in sum in the range from 10 ppm to 0.2% by weight, determined by gravimetric analysis.

Preferred inventive solid detergent compositions for hard surface cleaning and preferred inventive solid laundry detergent compositions may contain one or more surfactant, preferably one or more non-ionic surfactant.

Preferred non-ionic surfactants are alkoxylated alcohols, di-and multiblock copolymers of ethylene oxide and propylene oxide and reaction products of sorbitan with ethylene oxide or propylene oxide, alkyl polyglycosides (APG), hydroxyalkyl mixed ethers and amine oxides.

Preferred examples of alkoxylated alcohols and alkoxylated fatty alcohols are, for example, compounds of the general formula (III)

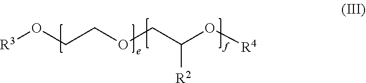

in which the variables are defined as follows:
R² is identical or different and selected from hydrogen and linear C₁-C₁₀-alkyl, preferably in each case identical and ethyl and particularly preferably hydrogen or methyl,
R³ is selected from C₈-C₂₂-alkyl, branched or linear, for example n-C₈H₁₇, n-C₁₀H₂₁, n-C₁₂H₂₅, n-C₁₄H₂₉, n-C₁₆H₃₃ or n-C₁₈H₃₇,
R⁴ is selected from C₁-C₁₀-alkyl, methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, n-heptyl, n-octyl, 2-ethylhexyl, n-nonyl, n-decyl or isodecyl,

The variables e and f are in the range from zero to 300, where the sum of e and f is at least one, preferably in the range of from 3 to 50. Even more preferably, e is in the range from 1 to 100 and f is in the range from 0 to 30.

In one embodiment, compounds of the general formula (III) may be block copolymers or random copolymers, preference being given to block copolymers.

Other preferred examples of alkoxylated alcohols are, for example, compounds of the general formula (IV)

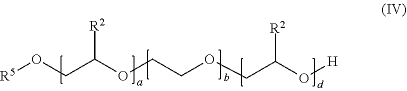

in which the variables are defined as follows:
R² is identical or different and selected from hydrogen and linear C₁-C₁₀-alkyl, preferably identical in each case and ethyl and particularly preferably hydrogen or methyl,
R⁵ is selected from C₆-C₂₀-alkyl, branched or linear, in particular n-C₈H₁₇, n-C₁₀H₂₁, n-C₁₂H₂₅, n-C₁₃H₂₇, n-C₁₅H₃₁, n-C₁₄H₂₉, n-C₁₆H₃, n-C₁₈H₃₇,
a is a number in the range from zero to 10, preferably from 1 to 6,
b is a number in the range from 1 to 80, preferably from 4 to 20,
d is a number in the range from zero to 50, preferably 4 to 25.

The sum a+b+d is preferably in the range of from 5 to 100, even more preferably in the range of from 9 to 50.

Preferred examples for hydroxyalkyl mixed ethers are compounds of the general formula (V)

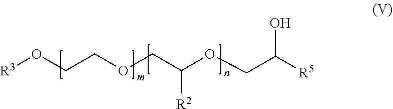

in which the variables are defined as follows:
R² is identical or different and selected from hydrogen and linear C₁-C₁₀-alkyl, preferably in each case identical and ethyl and particularly preferably hydrogen or methyl,
R³ is selected from C₈-C₂₂-alkyl, branched or linear, for example iso-C₁₁H₂₃, iso-C₁₃H₂₇, n-C₈H₁₇, n-C₁₀H₂₁, n-C₁₂H₂₅, n-C₁₄H₂₉, n-C₁₆H₃₃ or n-C₁₈H₃₇,
R⁵ is selected from C₆-C₂₀-alkyl, for example n-hexyl, isohexyl, sec-hexyl, n-heptyl, n-octyl, 2-ethylhexyl, n-nonyl, n-decyl, isodecyl, n-dodecyl, n-tetradecyl, n-hexadecyl, and n-octadecyl.

The variables m and n are in the range from zero to 300, where the sum of n and m is at least one, preferably in the range of from 5 to 50. Preferably, m is in the range from 1 to 100 and n is in the range from 0 to 30.

Compounds of the general formula (IV) and (V) may be block copolymers or random copolymers, preference being given to block copolymers.

Further suitable non-ionic surfactants are selected from di- and multiblock copolymers, composed of ethylene oxide and propylene oxide. Further suitable non-ionic surfactants are selected from ethoxylated or propoxylated sorbitan esters. Amine oxides or alkyl polyglycosides, especially linear C₄-C₁₆-alkyl polyglucosides and branched C₈-C₁₄-alkyl polyglycosides such as compounds of general average formula (VI) are likewise suitable.

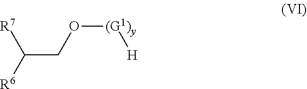

wherein:
R⁶ is C₁-C₄-alkyl, in particular ethyl, n-propyl or isopropyl,
R⁷ is —(CH₂)₂—R⁶,
G¹ is selected from monosaccharides with 4 to 6 carbon atoms, especially from glucose and xylose,
y in the range of from 1.1 to 4, y being an average number,

Further examples of non-ionic surfactants are compounds of general formula (VII) and (VIII)

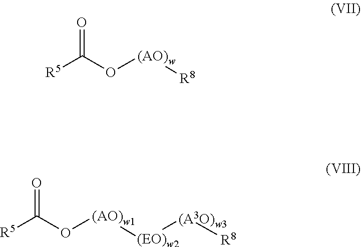

AO is selected from ethylene oxide, propylene oxide and butylene oxide,

EO is ethylene oxide, CH₂CH₂—O,

R⁸ selected from C₈-C₁₈-alkyl, branched or linear, and R⁵ is defined as above.

A³O is selected from propylene oxide and butylene oxide,
W is a number in the range of from 15 to 70, preferably 30 to 50,
w1 and w3 are numbers in the range of from 1 to 5, and
w2 is a number in the range of from 13 to 35.

An overview of suitable further non-ionic surfactants can be found in EP-A 0 851 023 and in DE-A 198 19 187.

Mixtures of two or more different non-ionic surfactants selected from the foregoing may also be present.

Other surfactants that may be present are selected from amphoteric (zwitterionic) surfactants and anionic surfactants and mixtures thereof.

Examples of amphoteric surfactants are those that bear a positive and a negative charge in the same molecule under use conditions. Preferred examples of amphoteric surfactants are so-called betaine-surfactants. Many examples of betaine-surfactants bear one quaternized nitrogen atom and one carboxylic acid group per molecule. A particularly preferred example of amphoteric surfactants is cocamidopropyl betaine (lauramidopropyl betaine).

Examples of amine oxide surfactants are compounds of the general formula (IX)

R⁹R¹⁰R¹¹N→O (IX)

wherein R⁹, R¹⁰, and R¹¹ are selected independently from each other from aliphatic, cycloaliphatic or C₂-C₄-alkylene C₁₀-C₂₀-alkylamido moieties. Preferably, R⁹ is selected from C₈-C₂₀-alkyl or C₂-C₄-alkylene C₁₀-C₂₀-alkylamido and R¹⁰ and R¹¹ are both methyl.

A particularly preferred example is lauryl dimethyl aminoxide, sometimes also called lauramine oxide. A further particularly preferred example is cocamidylpropyl dimethylaminoxide, sometimes also called cocamidopropylamine oxide.

Examples of suitable anionic surfactants are alkali metal and ammonium salts of C₈-C₁₈-alkyl sulfates, of C₈-C₁₈-fatty alcohol polyether sulfates, of sulfuric acid half-esters of ethoxylated C₄-C₁₂-alkylphenols (ethoxylation: 1 to 50 mol of ethylene oxide/mol), C₁₂-C₁₈ sulfo fatty acid alkyl esters, for example of C₁₂-C₁₈ sulfo fatty acid methyl esters, furthermore of C₁₂-C₁₈-alkylsulfonic acids and of C₁₀-C₁₈-alkylarylsulfonic acids. Preference is given to the alkali metal salts of the aforementioned compounds, particularly preferably the sodium salts.

Further examples for suitable anionic surfactants are soaps, for example the sodium or potassium salts of stearic acid, oleic acid, palmitic acid, ether carboxylates, and alkylether phosphates.

Preferably, inventive laundry detergent compositions contain at least one anionic surfactant.

In one embodiment of the present invention, inventive solid laundry detergent compositions may contain 0.1 to 60% by weight of at least one surfactant, selected from anionic surfactants, amphoteric surfactants and amine oxide surfactants.

In one embodiment of the present invention, inventive solid detergent compositions for cleaners may contain 0.1 to 60% by weight of at least one surfactant, selected from anionic surfactants, amphoteric surfactants and amine oxide surfactants.

In a preferred embodiment, inventive solid detergent compositions for cleaners and especially those for automatic dishwashing do not contain any anionic surfactant.

Inventive solid detergent compositions for hard surface cleaning and inventive solid laundry detergent compositions may contain at least one bleaching agent, also referred to as bleach. Bleaching agents may be selected from chlorine bleach and peroxide bleach, and peroxide bleach may be selected from inorganic peroxide bleach and organic peroxide bleach. Preferred are inorganic peroxide bleaches, selected from alkali metal percarbonate, alkali metal perborate and alkali metal persulfate.

Examples of organic peroxide bleaches are organic percarboxylic acids, especially organic percarboxylic acids.

In inventive solid detergent compositions for hard surface cleaning and in inventive solid laundry detergent compositions, alkali metal percarbonates, especially sodium percarbonates, are preferably used in coated form. Such coatings may be of organic or inorganic nature. Examples are glycerol, sodium sulfate, silicate, sodium carbonate, and combinations of at least two of the foregoing, for example combinations of sodium carbonate and sodium sulfate.

Suitable chlorine-containing bleaches are, for example, 1,3-dichloro-5,5-dimethylhydantoin, N-chlorosulfamide, chloramine T, chloramine B, sodium hypochlorite, calcium hypochlorite, magnesium hypochlorite, potassium hypochlorite, potassium dichloroisocyanurate and sodium dichloroisocyanurate.

Inventive solid detergent compositions for hard surface cleaning and inventive solid laundry detergent compositions may comprise, for example, in the range from 3 to 10% by weight of chlorine-containing bleach.

Inventive solid detergent compositions for hard surface cleaning and inventive solid laundry detergent compositions may comprise one or more bleach catalysts. Bleach catalysts can be selected from bleach-boosting transition metal salts or transition metal complexes such as, for example, manganese-, iron-, cobalt-, ruthenium- or molybdenum-salen complexes or carbonyl complexes. Manganese, iron, cobalt, ruthenium, molybdenum, titanium, vanadium and copper complexes with nitrogen-containing tripod ligands and also cobalt-, iron-, copper-and ruthenium-amine complexes can also be used as bleach catalysts.

Inventive solid detergent compositions for hard surface cleaning and inventive solid laundry detergent compositions may comprise one or more bleach activators, for example N-methylmorpholinium-acetonitrile salts (“MMA salts”), trimethylammonium acetonitrile salts, N-acylimides such as, for example, N-nonanoylsuccinimide, 1,5-diacetyl-2,2-dioxohexahydro-1,3,5-triazine (“DADHT”) or nitrile quats (trimethylammonium acetonitrile salts). Further examples of suitable bleach activators are tetraacetylethylenediamine (TAED) and tetraacetylhexylenediamine.

Inventive solid detergent compositions for hard surface cleaning and inventive solid laundry detergent compositions may comprise one or more corrosion inhibitors. In the present case, this is to be understood as including those compounds which inhibit the corrosion of metal. Examples of suitable corrosion inhibitors are triazoles, in particular benzotriazoles, bisbenzotriazoles, aminotriazoles, alkylaminotriazoles, also phenol derivatives such as, for example, hydroquinone, pyrocatechol, hydroxyhydroquinone, gallic acid, phloroglucinol or pyrogallol.

In one embodiment of the present invention, inventive solid detergent compositions for hard surface cleaning and inventive solid laundry detergent compositions comprise in total in the range from 0.1 to 1.5% by weight of corrosion inhibitor.

Inventive solid detergent compositions for hard surface cleaning and inventive solid laundry detergent compositions may comprise one or more builders, selected from organic and inorganic builders. Examples of suitable inorganic builders are sodium sulfate or sodium carbonate or silicates, in particular sodium disilicate and sodium metasilicate, zeolites, sheet silicates, in particular those of the formula α-Na₂Si₂O₅, β-Na₂Si₂O₅, and δ-Na₂Si₂O₅, also fatty acid sulfonates, α-hydroxypropionic acid, alkali metal malonates, fatty acid sulfonates, alkyl and alkenyl disuccinates, tartaric acid diacetate, tartaric acid monoacetate, oxidized starch, and polymeric builders, for example polycarboxylates and polyaspartic acid.

Examples of organic builders are especially polymers and copolymers. In one embodiment of the present invention, organic builders are selected from polycarboxylates, for example alkali metal salts of (meth)acrylic acid homopolymers or (meth)acrylic acid copolymers.

Suitable comonomers are monoethylenically unsaturated dicarboxylic acids such as maleic acid, fumaric acid, maleic anhydride, itaconic acid and citraconic acid. A suitable polymer is in particular polyacrylic acid, which preferably has an average molecular weight M_{w} in the range from 2000 to 40 000 g/mol, preferably 2000 to 10 000 g/mol, in particular 3000 to 8000 g/mol. Also of suitability are copolymeric polycarboxylates, in particular those of acrylic acid with methacrylic acid and of acrylic acid or methacrylic acid with maleic acid and/or fumaric acid, and in the same range of molecular weight.

It is also possible to use copolymers of at least one monomer from the group consisting of monoethylenically unsaturated C₃-C₁₀-mono- or C₄-C₁₀-dicarboxylic acids or anhydrides thereof, such as maleic acid, maleic anhydride, acrylic acid, methacrylic acid, fumaric acid, itaconic acid and citraconic acid, with at least one hydrophilic or hydrophobic monomer as listed below.

Suitable hydrophobic monomers are, for example, isobutene, diisobutene, butene, pentene, hexene and styrene, olefins with 10 or more carbon atoms or mixtures thereof, such as, for example, 1-decene, 1-dodecene, 1-tetradecene, 1-hexadecene, 1-octadecene, 1-eicosene, 1-docosene, 1-tetracosene and 1-hexacosene, C₂₂-α-olefin, a mixture of C₂₀-C₂₄-α-olefins and polyisobutene having on average 12 to 100 carbon atoms per molecule.

Suitable hydrophilic monomers are monomers with sulfonate or phosphonate groups, and also non-ionic monomers with hydroxyl function or alkylene oxide groups. By way of example, mention may be made of: allyl alcohol, isoprenol, methoxypolyethylene glycol (meth)acrylate, methoxypolypropylene glycol (meth)acrylate, methoxypolybutylene glycol (meth)acrylate, methoxypoly (propylene oxide-co-ethylene oxide) (meth)acrylate, ethoxypolyethylene glycol (meth)acrylate, ethoxypolypropylene glycol (meth)acrylate, ethoxypolybutylene glycol (meth)acrylate and ethoxypoly (propylene oxide-co-ethylene oxide) (meth)acrylate. Polyalkylene glycols here may comprise 3 to 50, in particular 5 to 40 and especially 10 to 30 alkylene oxide units per molecule.

Particularly preferred sulfonic-acid-group-containing monomers here are 1-acrylamido-1-propanesulfonic acid, 2-acrylamido-2-propanesulfonic acid, 2-acrylamido-2-methylpropanesulfonic acid, 2-methacrylamido-2-methylpropanesulfonic acid, 3-methacrylamido-2-hydroxypropanesulfonic acid, allylsulfonic acid, methallylsulfonic acid, allyloxybenzenesulfonic acid, methallyloxybenzenesulfonic acid, 2-hydroxy-3-(2-propenyloxy) propanesulfonic acid, 2-methyl-2-propene-1-sulfonic acid, styrenesulfonic acid, vinylsulfonic acid, 3-sulfopropyl acrylate, 2-sulfoethyl methacrylate, 3-sulfopropyl methacrylate, sulfomethacrylamide, sulfomethylmethacrylamide, and salts of said acids, such as sodium, potassium or ammonium salts thereof.

Particularly preferred phosphonate-group-containing monomers are vinylphosphonic acid and its salts.

A further example of builders is carboxymethyl inulin.

Moreover, amphoteric polymers can also be used as builders.

Inventive solid detergent compositions for hard surface cleaning and inventive solid laundry detergent compositions may comprise, for example, in the range from in total 10 to 70% by weight, preferably up to 50% by weight, of builder. In the context of the present invention, (A1) and (A2) are not counted as builder.

In one embodiment of the present invention, inventive solid detergent compositions for hard surface cleaning and inventive solid laundry detergent compositions may comprise one or more cobuilders.

Inventive solid detergent compositions for hard surface cleaning and inventive solid laundry detergent compositions may comprise one or more antifoams, selected for example from silicone oils and paraffin oils.

In one embodiment of the present invention, inventive solid detergent compositions for hard surface cleaning and inventive solid laundry detergent compositions comprise in total in the range from 0.05 to 0.5% by weight of antifoam.

Inventive solid detergent compositions for hard surface cleaning and inventive solid laundry detergent compositions may comprise one or more enzymes. Examples of enzymes are lipases, hydrolases, amylases, proteases, cellulases, esterases, pectinases, lactases and peroxidases.

In one embodiment of the present invention, inventive solid detergent compositions for hard surface cleaning and inventive solid laundry detergent compositions may comprise, for example, up to 5% by weight of enzyme, preference being given to 0.1 to 3% by weight. Said enzyme may be stabilized, for example with the sodium salt of at least one C₁-C₃-carboxylic acid or C₄-C₁₀-dicarboxylic acid. Preferred are formates, acetates, adipates, and succinates.

In one embodiment of the present invention, inventive solid detergent compositions for hard surface cleaning and inventive solid laundry detergent compositions comprise at least one zinc salt. Zinc salts can be selected from water-soluble and water-insoluble zinc salts. In this connection, within the context of the present invention, water-insoluble is used to refer to those zinc salts which, in distilled water at 25° C., have a solubility of 0.1 g/l or less. Zinc salts which have a higher solubility in water are accordingly referred to within the context of the present invention as water-soluble zinc salts.

In one embodiment of the present invention, zinc salt is selected from zinc benzoate, zinc gluconate, zinc lactate, zinc formate, ZnCl₂, ZnSO₄, zinc acetate, zinc citrate, Zn(NO₃)₂, Zn(CH₃SO₃)₂ and zinc gallate, preferably ZnCl₂, ZnSO₄, zinc acetate, zinc citrate, Zn(NO₃)₂, Zn(CH₃SO₃)₂ and zinc gallate.

In another embodiment of the present invention, zinc salt is selected from ZnO, ZnO·aq, Zn(OH)₂ and ZnCO₃. Preference is given to ZnO·aq.

In one embodiment of the present invention, zinc salt is selected from zinc oxides with an average particle diameter (weight-average) in the range from 10 nm to 100 μm.

The cation in zinc salt can be present in complexed form, for example complexed with ammonia ligands or water ligands, and in particular be present in hydrated form. To simplify the notation, within the context of the present invention, ligands are generally omitted if they are water ligands.

Depending on how the pH of mixture according to the invention is adjusted, zinc salt can change. Thus, it is for example possible to use zinc acetate or ZnCl₂ for preparing formulation according to the invention, but this converts at a pH of 8 or 9 in an aqueous environment to ZnO, Zn(OH)₂ or ZnO·aq, which can be present in non-complexed or in complexed form.

Zinc salt may be present in those detergent compositions for cleaners according to the invention which are solid at ambient temperature are preferably present in the form of particles which have for example an average diameter (number-average) in the range from 10 nm to 100 μm, preferably 100 nm to 5 μm, determined for example by X-ray scattering.

Zinc salt may be present in those detergent compositions for home which are liquid at ambient temperature in dissolved or in solid or in colloidal form.

In one embodiment of the present invention, detergent compositions for cleaners and laundry detergent compositions comprise in total in the range from 0.05 to 0.4% by weight of zinc salt, based in each case on the solids content of the composition in question.

Here, the fraction of zinc salt is given as zinc or zinc ions. From this, it is possible to calculate the counterion fraction.

In one embodiment of the present invention, inventive solid detergent compositions for hard surface cleaning and inventive solid laundry detergent compositions are free from heavy metals apart from zinc compounds. Within the context of the present, this may be understood as meaning that detergent compositions for cleaners and laundry detergent compositions according to the invention are free from those heavy metal compounds which do not act as bleach catalysts, in particular of compounds of iron and of bismuth. Within the context of the present invention, “free from” in connection with heavy metal compounds is to be understood as meaning that the content of heavy metal compounds which do not act as bleach catalysts is in sum in the range from 0 to 100 ppm, determined by the leach method and based on the solids content. Preferably, formulation according to the invention has, apart from zinc, a heavy metal content below 0.05 ppm, based on the solids content of the formulation in question. The fraction of zinc is thus not included.

Within the context of the present invention, “heavy metals” are defined to be any metal with a specific density of at least 6 g/cm³ with the exception of zinc. In particular, the heavy metals are metals such as bismuth, iron, copper, lead, tin, nickel, cadmium and chromium.

Preferably, inventive solid detergent compositions for hard surface cleaning and inventive solid laundry detergent compositions comprise no measurable fractions of bismuth compounds, i.e. for example less than 1 ppm.

In one embodiment of the present invention, inventive solid detergent compositions for hard surface cleaning and inventive solid laundry detergent compositions comprise one or more further ingredient such as fragrances, dyestuffs, organic solvents, buffers, disintegrants for tablets (“tabs”), and/or acids such as methylsulfonic acid.

Preferred example detergent compositions for automatic dishwashing may be selected according to table 1.

TABLE 1
Example detergent compositions for automatic dishwashing

| All amounts in g/sample | ADW.1 | ADW.2 | ADW.3 |
|---|---|---|---|
| Granule according to example II | 30 | 22.5 | 15 |
| Protease | 2.5 | 2.5 | 2.5 |
| Amylase | 1 | 1 | 1 |
| n—C₁₈H₃₇—O(CH₂CH₂O)₉H | 5 | 5 | 5 |
| Polyacrylic acid M_{w} 4000 g/mol as | 10 | 10 | 10 |
| sodium salt, completely neutralized | | | |
| Sodium percarbonate | 10.5 | 10.5 | 10.5 |
| TAED | 4 | 4 | 4 |
| Na₂Si₂O₅ | 2 | 2 | 2 |
| Na₂CO₃ | 19.5 | 19.5 | 19.5 |
| Sodium citrate dihydrate | 15 | 22.5 | 30 |
| HEDP | 0.5 | 0.5 | 0.5 |
| ethoxylated polyethylenimine, 20 | optionally: | optionally: | optionally: |
| EO/NH group, Mₙ: 30,000 g/mol | 0.1 | 0.1 | 0.1 |

Laundry detergent compositions according to the invention are useful for laundering any type of laundry, and any type of fibres. Fibres can be of natural or synthetic origin, or they can be mixtures of natural of natural and synthetic fibres. Examples of fibers of natural origin are cotton and wool. Examples for fibers of synthetic origin are polyurethane fibers such as Spandex® or Lycra®, polyester fibers, or polyamide fibers. Fibers may be single fibers or parts of textiles such as knitwear, wovens, or nonwovens.

Another aspect of the present invention is a process for making tablets for automatic dishwashing from a powder or granule, wherein said granule or powder is selected from inventive granules and inventive powders, respectively. Said process is hereinafter also referred to as pelletizing process according to the invention.

Inventive tablets are preferably made with the help of a machine, for example a tablet press.

The pelletizing process according to the invention can be carried out by mixing inventive granule or powder with at least one non-ionic surfactant and optionally one or more further substance and then compressing the mixture to give tablets. Examples of suitable non-ionic surfactants and further substances such as builders, enzymes are listed above. Particularly preferred examples of non-ionic surfactants are hydroxy mixed ethers, for example hydroxy mixed ethers of the general formula (V).

The invention is further illustrated by working examples.

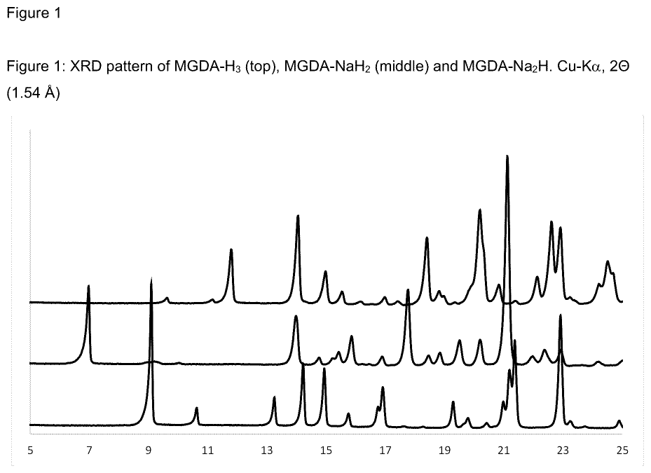

Brief description of the drawing: FIG. 1: XRD pattern of MGDA-H₃ (top), MGDA-NaH₂ (middle) and MGDA-Na₂H. Cu—Kα, 2Θ (1.54 Å).

X-ray diffraction (XRD) data was collected as powder X-ray using a diffractometer (D8 Advance Series II, Bruker AXS GmbH) equipped with a LYNXEYE detector operated with a Copper anode X-ray tube running at 40 KV and 40 mA. The geometry was Bragg-Brentano, and air scattering was reduced using an air scatter shield.

Data collection: Samples were homogenized in a mortar and then pressed into a standard flat sample holder provided by Bruker AXS GmbH for Bragg-Brentano geometry data collection. The flat surface was achieved using a glass plate to compress and flatten the sample powder. The data was collected from the angular range 2 to 80° 2Theta with a step size of 0.02° 2Theta while the variable divergence slit was set to a fixed angle of 0.1°. The peak position and intensity determination was performed using DIFFRAC.EVA. (User Manual for DIFFRAC.EVA, Bruker AXS GmbH, Karlsruhe.)

The following tables lists the reflections associated with the given substances. The reflections from the NaCl crystals were neglected.

General: Percentages refer to percent by weight unless specifically indicated otherwise.

### STARTING MATERIALS

Solution (A.1): 40% by weight aqueous solution of the trisodium salt of methylglycine diacetic acid (MGDA-Na₃).

Granule (A.1): Experiment 5 of WO 2017/220308 was repeated. A granule of MGDA-Na₃, diameter 350 to 1000 μm, average particle diameter (d50) 725 μm, determined by sieving, also referred to as C-Gr.3. C-Gr.3 was obtained.

Complexing agent (B.1): methylglycine diacetic acid (MGDA-H₃) as powder and with a purity of about than 72%, determined by iron binding capacity, remainder: sodium sulfate.

Complexing agent (B.2): methylglycine diacetic acid (MGDA-H₃) as powder with a purity of about than 85%, determined by iron binding capacity, remainder: sodium chloride.

Complexing agent (B.3): methylglycine diacetic acid (MGDA-H₃) as powder with a purity of about than 95%, determined by iron binding capacity

Complexing agent (B.4): methylglycine diacetic acid (MGDA-H₃) as powder with a purity of about than 80%, determined by iron binding capacity, remainder: sodium chloride.

Complexing agent (B.5): methylglycine diacetic acid (MGDA-HNa₂) as powder with a of about than 84%, determined by iron binding capacity, remainder: NaCl.

The iron binding capacity was determined by potentiometric FeCl₃ titration.

### Manufacture of (B.1)

A 3-litre vessel made of glass equipped with overhead stirrer and temperature measuring device was charged with 1000 g of solution (A.1) at ambient temperature. Then, 226 g of sulfuric acid (96 wt %) were added slowly under stirring. During addition the temperature was not allowed to rise above 50° C., by adjustment of dosing speed and active cooling via water/NaCl bath.

After complete H₂SO₄ addition (pH=0,8; determined as 1 wt % solution in water) crystallization could be observed and the resulting suspension was stirred at ambient temperature and normal pressure for 60 to 120 minutes, then filtered over a suction filter and the filter cake washed three times with 100 ml of water (5° C.). The wet filter cake was dried for 12 h at 40° C. to yield 140 g of white crystalline powder (B.1).

### Manufacture of (B.2)

A 3-litre vessel made of glass equipped with overhead stirrer and temperature measuring device was charged with 750 g of solution (A.1) at ambient temperature. Then, 483 g of aqueous hydrochloric acid (30 wt %) were added slowly under stirring. During addition the temperature was not allowed to rise above 50° C., by adjustment of dosing speed and active cooling via water/NaCl bath.

After complete HCl addition (pH=0,4; determined as 1 wt % solution in water) the resulting solution was transferred in a flask and concentrated at 70° C. and 20 mbar until crystallization could be observed. The resulting suspension was stirred at ambient temperature and normal pressure for 30 to 60 minutes, then filtered over a suction filter and the filter cake washed three times with 100 mL of cold water (5° C.). The wet filter cake was dried for 12 h at 40° C. to yield 162 g of white crystalline powder (B.2).

### Manufacture of (B.3)

A 3-litre vessel made of glass equipped with overhead stirrer and temperature measuring device was charged with 120 g of complexing agent (B.2) and 1000 g of deionized water and stirred at 50° C. until a clear solution was obtained. The resulting solution was transferred in a flask and concentrated at 70° C. and 20 mbar until crystallization could be observed. The resulting suspension was stirred at ambient temperature and normal pressure for 60 to 120 minutes, then filtered over a suction filter and the filter cake washed three times with 50 mL of cold water (5° C.). The wet filter cake was dried for 12 h at 40° C. to yield 42 g of white crystalline powder (B.3).

### Manufacture of (B.4)

160 g of white crystalline powder (B.4) were obtained following the same method as (B.2) but using 1000 g of solution (A.1) and 337 g of hydrochloric acid (30 wt %). pH value after HCl addition was 2.3 (determined as 1 wt % solution in water).

### Manufacture of (B.5)

283 g of white crystalline powder (B.5) were obtained following the same method as (B.2) but using 1000 g of solution (A.1) and 193 g of hydrochloric acid (30 wt %). pH value after HCl addition was 8.0 (determined as 1 wt % solution in water).

### I. MANUFACTURE OF INVENTIVE GRANULES

### I.1 Manufacture of Spray Liquors

### I.1.1 Manufacture of Spray Liquor SL. 1

A vessel was charged with 265 g of demineralized water and 35 g of complexing agent (B.1) was added. Spray liquor SL. 1 was obtained. was stirred vigorously. and then heated to 70° C. for 3 hours and then subjected to spray granulation.

### I.1.2 Manufacture of Spray Liquor SL.2

A vessel was charged with 268 g of demineralized water and 32 g of complexing agent (B.2) was added. The spray liquor SL.2 so obtained was stirred vigorously. and then heated to 70° C. for 3 hours and then subjected to spray granulation.

### I.2. Spray Granulation

### I.2.1 Manufacture of Inventive Granule Gr. 1

Step (a.1): A lab scale granulator, commercially available as “WFP-Mini” from the company DMR, was charged with 300 g of granule (A.1).

An amount of 22 Nm³/h of nitrogen with a temperature of 130-160° C. was blown from the bottom. A fluidized bed of (A.1) was obtained.

Step (b.1), Simultaneously with Step (c.1):

Spray liquor SL.1 was heated to 70° C. for 3 hours under vigorous stirring and then introduced into the spray granulator by spraying 5 to 7 g/minute SL.1 (about 22° C.) into the fluidized bed of (A.1) from step (a.1) from the bottom through a three-fluid nozzle.

The pressure of the atomizing gas was 1.5 to 2.0 bar, abs. Granule (A.1) was coated, and the bed temperature, which corresponds to the surface temperature of the solids in the fluidized bed, was 95-105° C.

When SL.1 was used up, the granule obtained was cooled down. inventive granule Gr. 1 was recovered.

### I.2.2 Manufacture of Inventive Granule Gr.2

Step (a.2): A lab scale granulator, commercially available as “WFP-Mini” from the company DMR, was charged with 300 g of granule (A.1).

An amount of 22 Nm³/h of nitrogen with a temperature of 130 to 160° C. was blown from the bottom. A fluidized bed of (A.1) was obtained.

Step (b.2), Simultaneously with Step (c.2):

Spray liquor SL.2 was heated to 70° C. for 3 hours under vigorous stirring and then introduced into the spray granulator by spraying 5 to 7 g/minute SL.2 (about 22° C.) into the fluidized bed of

(A.1) from step (a.2) from the bottom through a three-fluid nozzle.

The pressure of the atomizing gas was 1.5 to 2.0 bar, abs. Granule (A.1) was coated, and the bed temperature, which corresponds to the surface temperature of the solids in the fluidized bed, was 95 to 105° C.

When SL.2 was used up, the granule obtained was cooled down. Granule Gr.2 were recovered as inventive granules.

In the above examples, nitrogen can be replaced by air having the same temperature.

### I.2.3 Manufacture of a Comparative Granule C-Gr.4

Example 1 of U.S. Pat. No. 8,940,678 was followed. C-Gr.4 was obtained.

II. TESTING OF INVENTIVE GRANULES AND OF COMPARATIVE GRANULE

Test protocol: 10 g of inventive granule or of C-Gr.3 were mixed with 5 g Na-percarbonate and placed in a vial having a permeable stopper to allow an exchange with the surrounding atmosphere. The vial was stored for 28 days in a climate-chamber at 35° C. and 70% humidity.

The discoloration of the above stored mixtures was determined by measuring the b-value of the CIELAB color space (Mach 5 measurement).

TABLE 2
Yellowing behavior of inventive granules
and of comparative granule C-Gr.3

| | b-value (start) | b-value (14 days) | b-value (28 days) |
|---|---|---|---|
| Gr.1 | 2.1 | 7.9 | 21.7 |
| Gr.2 | 2.7 | 10.7 | 23.9 |
| C-Gr.3 | 3.1 | 13.1 | 27.8 |

The XRD patterns of Gr.1 and Gr.2 exhibited a peak at 2Θ=14.06 and 18.41 and 26.82, determined with Cu—Kα radiation (1.54 Å). The XRDs spectrum of C-Gr.3 exhibited neither peak at 2Θ=9.1 and a duplet peak at 2Θ14.23 and 14.95, nor peaks at 2Θ of 6.98, 13.99 and 17.77, nor peaks at 2Θ of 14.06 and 18.41 and 26.82.

TABLE 3
XRD data (all)

| MGDA—Na₂H | | | MGDA—NaH₂ | | | MGDA—H₃ | | |
|---|---|---|---|---|---|---|---|---|
| | | rel. | | | rel. | | | rel. |
| | | Intensity | | | Intensity | | | Intensity |
| Angle | d Value | [%] | Angle | d Value | [%] | Angle | d Value | [%] |
| 9.10 | 9.71 | 100 | 9.10 | 9.71 | 100 | 11.81 | 7.49 | 56 |
| 10.64 | 8.31 | 12 | 10.64 | 8.31 | 12 | 14.06 | 6.29 | 93 |
| 13.26 | 6.67 | 20 | 13.26 | 6.67 | 20 | 14.99 | 5.91 | 35 |
| 14.23 | 6.22 | 46 | 14.23 | 6.22 | 46 | 18.41 | 4.81 | 72 |
| 14.95 | 5.92 | 43 | 14.95 | 5.92 | 43 | 20.20 | 4.39 | 100 |
| 15.76 | 5.62 | 10 | 15.76 | 5.62 | 10 | 20.26 | 4.38 | 66 |
| 16.76 | 5.28 | 15 | 16.76 | 5.28 | 15 | 20.84 | 4.26 | 21 |
| 16.92 | 5.24 | 29 | 16.92 | 5.24 | 29 | 22.14 | 4.01 | 30 |
| 19.30 | 4.60 | 20 | 19.30 | 4.60 | 20 | 22.62 | 3.93 | 90 |
| 19.80 | 4.48 | 7 | 19.80 | 4.48 | 7 | 22.92 | 3.88 | 83 |
| 20.99 | 4.23 | 20 | 20.99 | 4.23 | 20 | 24.22 | 3.67 | 22 |
| 21.20 | 4.19 | 43 | 21.20 | 4.19 | 43 | 24.52 | 3.63 | 46 |
| 21.38 | 4.15 | 69 | 21.38 | 4.15 | 69 | 24.70 | 3.60 | 33 |
| 22.92 | 3.88 | 86 | 22.92 | 3.88 | 86 | | | |

TABLE 4
characteristic XRD data (selection)

| MGDA—Na₂H | | MGDA—NaH₂ | | MGDA—H₃ | |
|---|---|---|---|---|---|
| Angle | d Value | Angle | d Value | Angle | d Value |
| 9.10 | 9.71 | 6.98 | 12.65 | 14.06 | 6.29 |
| 14.23 | 6.22 | 13.99 | 6.32 | 18.41 | 4.81 |
| 14.95 | 5.92 | 17.77 | 4.99 | 26.82 | 3.32 |

Table 5: Water uptake during storage due to hygroscopicity

The hygroscopicity was determined by storing at 25° C. and 50% relative humidity over a period of 48 hours. In the alternative, so-called tropic conditions are storing at 35° C. and 70 to 90% relative humidity over a period of 24 hours. Flowability grades: from zero (free flowable granule/powder) to 4 (granule/powder has dissolved). The grades and the water content (Karl-Fischer titration) were determined.

| | Humidity [% by weight] | | | | Flowability |
|---|---|---|---|---|---|
| | After 1 | After 8 | after 24 | after 48 | grade |
| | hour | hours | hours | hours | after 48 h |
| Gr.1 | 4.0 | 11.6 | 13.3 | 14.7 | 2.5 |
| Gr.2 | 5.1 | 13.1 | 15.6 | 17.1 | 2.3 |
| C-Gr.3 | 3.2 | 12.5 | 18.7 | 22.3 | n.d. |
| C-Gr.4 | 5.7 | 27.7 | 54.2 | 68.7 | 4.0 |

n.d.: not determined

1. A process for making a modified granule or powder comprising an alkali metal salt of an aminocarboxylate complexing agent (A), said process comprising the steps of
(a) providing a granule or powder comprising an alkali metal salt of an aminocarboxylate complexing agent (A),
(b) treating said granule or powder with the aminocarboxylate complexing agent (A) in which at least one amino group or carboxyl group per molecule is present in the acid form, in the presence or absence of water, and
(c) performing a heat treatment at a temperature in the range of from 80 to 150° C.

2. The process according to claim 1, wherein step (b) is performed by spraying an aqueous solution or slurry of the aminocarboxylate complexing agent (A) in which at least one amino or carboxyl group is present in the acid form on said powder or granule, and wherein step (c) includes an at least partial removal of water by evaporation.

3. The process according to claim 1, wherein steps (b) and (c) are performed in a fluidized bed or in an essentially horizontal cylindrical drying apparatus containing a stirring element that rotates around an essentially horizontal axis.

4. The process according to claim 1, wherein the granule or powder of said aminocarboxylate complexing agent (A) in step (a) is a trialkali metal salt of methylglycine diacetic acid (MGDA).

5. The process according to claim 1, wherein the granule or powder of said aminocarboxylate complexing agent (A) in step (a) is a granule or powder from compounds according to general formula (I)
[CH₃—CH(COO)—N(CH₂—COO)₂]M₃₋ₓHₓ (I)
wherein
M is selected from alkali metal cations, same or different, and
x in formula (I) is in the range of from zero to 0.30.

6. The process according to claim 4, wherein methylglycine diacetate (MGDA) alkali metal salt (A) is selected from the racemic mixture or mixtures of the enantiomers with predominantly the L-enantiomer with an ee value in the range of from 0.1 to 20%.

7. The process according to claim 1, wherein the aminocarboxylate complexing agent (A) used in step (b) is selected from the free acid, mono- or dialkali metal salts, or combinations thereof.

8. A granule or powder of a solid alkali metal salt (A) of an aminocarboxylate complexing agent wherein said granule or powder has a pH value gradient, the pH value being higher in the core than at the surface.

9. A granule or powder of a solid alkali metal salt (A) of an aminocarboxylate complexing agent wherein (A) in the core is selected from compounds according to general formula (I)
[CH₃—CH(COO)—N(CH₂—COO)₂]M₃₋ₓHₓ (I)
wherein
M is selected from alkali metal cations, same or different, and
x is in the range of from zero to 0.3
and (A) at the surface is selected from the same compound but with x being in the range of from 1.0 to 3.0.

10. The granule according to claim 8, wherein said granule has an average particle diameter (d50) in the range of from 150 μm to 1.5 mm.

11. The powder according to claim 8, wherein said powder has an average particle diameter (d50) in the range of from 50 μm to 100 μm.

12. The granule or powder according to claim 8, wherein the XRD pattern of said powder or granule exhibits a peak at 2Θ=9.1 and a duplet peak at 2Θ14.23 and 14.95, or peaks at 2Θ=698, 13.99 and 17.77, or peaks at 2Θ=14.06 and 18.41 and 26.82, determined with Cu—Kα radiation.

13. A method for manufacturing a cleaning agent comprising using a powder or granule according to claim 8.

14. A method for manufacturing an automatic dishwashing detergent comprising using a powder or granule according to claim 8.

15. A cleaning agent comprising at least one powder or granule according to claim 8.

16. The granule according to claim 9, wherein said granule has an average particle diameter (d50) in the range of from 150 μm to 1.5 mm.

17. The granule according to claim 9, wherein said granule has an average particle diameter (d50) in the range of from 50 μm to 100 μm.

18. The granule or powder according to claim 9, wherein the XRD pattern of said powder or granule exhibits a peak at 2Θ=9.1 and a duplet peak at 2Θ 14.23 and 14.95, or peaks at 2Θ=698, 13.99 and 17.77, or peaks at 2Θ=14.06 and 18.41 and 26.82, determined with Cu-Ka radiation.

19. A method for manufacturing a cleaning agent or an automatic dishwashing detergent comprising using a powder or granule according to claim 9.

20. A cleaning agent comprising at least one powder or granule according to claim 9.
